SEQUENCE LISTING (12 sequences)

SEQ ID NO: 1 - DNA, 4132 bp, Sinipercachuatsi
  SNP molecular marker
aacaaagtacatttactacagtaagtacacttctgaggtactgtgtgtgtgtaaagtgggaaagtgagagtagaaacacagcggggctttctgtctttcactctctctgttttctctttcctgtctggaatacaagttcagcagcatgttggagacagcgacacacagcagagctcagtctgcagccaccatcaccaccaccaccaccatcatcaccgccaccatcttcatcatgttcagctggaccggcctgctcttcgtcctctgcaccctgactcctgcgctctgctgtgattggctcagacactacggtcatgtgagcaacagctctctgactctcgtccagatcatgtttgggggtccttaaagaggttcagggtacttcaacatgtgctcagagtctctgtcaggtcctcgagggggtttaaataattctttacacactatataagtttacatctatcaatgcatattaaatatatatatatacttacataaacataaaatagatcatgtatgttaacacatgtggacggatgttaaaaatagactctatatatttacatatacagacacatattgaatccaactcacgatgagaacgagactctgacctttctgaagtgggacgagcagaggaagcttcagattaagagtcagtgtgacaggaggaatgatcacagacaataataactctgtaacatacagacagacgtgagactgttagttacgtgagagagtcttaaaaaactctgcactggccctttaaggggctgtcgactcgttagtagcatctagtggagctttaagaaactacaacagcagaaacacctgtctcctccattacacaccacacaaagtctgacatatgtgcgtgtatcagcatatatgctgttaatggactgtatatgtttattaaaactttattatatgtttacatatgctgattataataaaacttgataatatatgtttacatatactgattataataaaactttattatatgtttacatatactgattataataaaactttattatatgtttacatatactgattataataaatctttattatatatgtttacatatattggtgtaaagttaaaaaaaagtttcattatatatttacatatatgaaccaatagttaaaaataaaaaaacatatctttacatatgtgtaaaaactcattcaggaaacgttcatacgcacagatttgatcttaacttgtgtgttcgagcaaatttagtattttgtcagatttctttcttattttaaaaatgtgtgtaggtaagaaaaataattaggagtggtccgtatcggtcaggacgtaaaggaaatgtttctcttgttcggtttttacaaacaaatttaagggaaatgaagaaaacattggtaaaagagccccaaagtttccaacatctagagtgtggcaccacttcaatttccagtggtttgtaaagtgtgtgatgtggtgaaatgaagtgacatcttattgtgtctttaatgtttcagggcggtcagatgactgaagaggagagtccattttcctttccaaacaaactctacaaacacatacaaaaggcagaggtaataaatctatcaatcgatctatctctctatgtgacatttcaactgctttcatccctcgctcttcaaatgctttcactgcttacatgtcgaactgacagtttcactgctttgtgctcttattcgttaactgcgagttgtgtgttttcagcgattacacttcaactgacacttcaactttactcagcgcttacacttaaattagcacttcaacttcatttagtgcttacatttaaactgaaacttatctcagcccttttatttcaactgccacttcaacttctttcagtatttcaaatgtttacctgcagattttcagccctgagcgcacatacatgtccgtcagataatctttctgtttctcgtcgtatattattattattattattcacgtttataacgacacactttattattattattattcacgtttataacgacacactttattattattattattcacgtttataacgacacactttattattattcatgtttataacgacacacttcattattattattattcactttaataacaacacactttattattattattcacacgtttataacgacacactttattatgattattcacgtttataacgacacactttattattattcatgtttataacgacacactttattattattattcacgttaataacgacacactttattattattattcacacgtttataacgacacactttattattattattcatgtttataacaacacactttattattattattcacgttaataacgacacactttattattattattcacacgtttataacgacacactttattatgattattcacgttaataacgacatgctttattattattaatcacgacttataacgacgcacttttattattattgattctcacggttataacgaccctgctttattatacaatcatataaatcaaactgcagctgtcttactgttatgatgatgacatcagctactgtcagcctattcataaaagctacgaaattcaccaccctgtggagtcccagctggttttcatcagagacagtctggagctaattcttggtctctatcgccatgacaacctctcctctgctacctgggataccgacaagacggagcacttcctgttgtgcatccacagacagatagatggactcaatagctgtgtgagtagacagacagagagacacttcaactcctttcagctctttacttcatctgatacttaaagtgctttcagtgcttatacttcagtcaacagttcaactgacatcttttcacacatggacttacatcaactcctttcagtgcttacacttcaactgccagtttaacttctttgagtacgtacacatcagctgaaacacttaaaaacacaacttaaacttcaaatgtgctgaaattgtgctcagacatcaactggcactaaacttattttagtgcttacactttttatacttcagtcaacgcttcaactgacatatttgagtttttacacgtggactgactcctttcatgacttacacttcaacttctagctccttttaccacttacacctcaagcaacactttaactccttgtgtgacacaacagcccttctacttccgtcattacatttttacacttgaacagacaccaactactttcaggacttagcctgcaactcacacttcaaactcttttgacaaattacactttaagtgacatttcaactgacacttaaagtccttttagtacttacactttctagtgacatgtaacgccttgtatttccccgaacatatcaagtgacttcttatgcttcaaacttcaagctcctttcactacttcaactgtcatgtcaatgcctttcactgcttacacttttccttttacatgttcaacttcaggttttgcctccaaccacagacaaacaatctgacctgtgtttttgtcttcaggtgtcgactaacaagcaagcaaactacaaactgaaaaaatactacaagagactggagaaaagcactctgtaccgcactgtgagtacaagcactgttgtagtactgactgtagttctgtgtacagggtgaaagtattggtactgactgtagttctgtgtacagggtgaaagtagtagtactgactgtagttctgtgtacagggtggtagtgctgcgtcctgggagctgatcaggaaggaaactgaagtgcacctggaacggtcggccctgctggtggcccccatagctacaacacttcatcaccaacactgacacgtctcatcagtctgtctcactgtctccagcgtttattctgtctctctgtgagagtttctattgtcagtctatttatttatttatcatttatcgtttctatttatgaacaaagtgctcatcttgtatttatttatttattgaactctgacagttttgtatttacagactctgagtctgattttacttgaaaaattttgataacggaccacaataaagacatttcatacaaaaaaaaaaaaaaaaaaaaaaaaaaaaaaaaa

SEQ ID NO: 2 - DNA, 4132 bp, Sinipercachuatsi
  SNP molecular marker
aacaaagtacatttactacagtaagtacacttctgaggtactgtgtgtgtgtaaagtgggaaagtgagagtagaaacacagcggggctttctgtctttcactctctctgttttctctttcctgtctggaatacaagttcagcagcatgttggagacagcgacacacagcagagctcagtctgcagccaccatcaccaccaccaccaccatcatcaccgccaccatcttcatcatgttcagctggaccggcctgctcttcgtcctctgcaccctgactcctgcgctctgctgtgattggctcagacactacggtcatgtgagcaacagctctctgactctcgtccagatcatgtttgggggtccttaaagaggttcggggtacttcaacatgtgctcagagtctctgtcaggtcctcgagggggtttaaataattctttacacactatataagtttacatctatcaatgcatattaaatatatatatatacttacataaacataaaatagatcatgtatgttaacacatgtggacggatgttaaaaatagactctatatatttacatatacagacacatattgaatccaactcacgatgagaacgagactctgacctttctgaagtgggacgagcagaggaagcttcagattaagagtcagtgtgacaggaggaatgatcacagacaataataactctgtaacatacagacagacgtgagactgttagttacgtgagagagtcttaaaaaactctgcactggccctttaaggggctgtcgactcgttagtagcatctagtggagctttaagaaactacaacagcagaaacacctgtctcctccattacacaccacacaaagtctgacatatgtgcgtgtatcagcatatatgctgttaatggactgtatatgtttattaaaactttattatatgtttacatatgctgattataataaaacttgataatatatgtttacatatactgattataataaaactttattatatgtttacatatactgattataataaaactttattatatgtttacatatactgattataataaatctttattatatatgtttacatatattggtgtaaagttaaaaaaaagtttcattatatatttacatatatgaaccaatagttaaaaataaaaaaacatatctttacatatgtgtaaaaactcattcaggaaacgttcatacgcacagatttgatcttaacttgtgtgttcgagcaaatttagtattttgtcagatttctttcttattttaaaaatgtgtgtaggtaagaaaaataattaggagtggtccgtatcggtcaggacgtaaaggaaatgtttctcttgttcggtttttacaaacaaatttaagggaaatgaagaaaacattggtaaaagagccccaaagtttccaacatctagagtgtggcaccacttcaatttccagtggtttgtaaagtgtgtgatgtggtgaaatgaagtgacatcttattgtgtctttaatgtttcagggcggtcagatgactgaagaggagagtccattttcctttccaaacaaactctacaaacacatacaaaaggcagaggtaataaatctatcaatcgatctatctctctatgtgacatttcaactgctttcatccctcgctcttcaaatgctttcactgcttacatgtcgaactgacagtttcactgctttgtgctcttattcgttaactgcgagttgtgtgttttcagcgattacacttcaactgacacttcaactttactcagcgcttacacttaaattagcacttcaacttcatttagtgcttacatttaaactgaaacttatctcagcccttttatttcaactgccacttcaacttctttcagtatttcaaatgtttacctgcagattttcagccctgagcgcacatacatgtccgtcagataatctttctgtttctcgtcgtatattattattattattattcacgtttataacgacacactttattattattattattcacgtttataacgacacactttattattattattattcacgtttataacgacacactttattattattcatgtttataacgacacacttcattattattattattcactttaataacaacacactttattattattattcacacgtttataacgacacactttattatgattattcacgtttataacgacacactttattattattcatgtttataacgacacactttattattattattcacgttaataacgacacactttattattattattcacacgtttataacgacacactttattattattattcatgtttataacaacacactttattattattattcacgttaataacgacacactttattattattattcacacgtttataacgacacactttattatgattattcacgttaataacgacatgctttattattattaatcacgacttataacgacgcacttttattattattgattctcacggttataacgaccctgctttattatacaatcatataaatcaaactgcagctgtcttactgttatgatgatgacatcagctactgtcagcctattcataaaagctacgaaattcaccaccctgtggagtcccagctggttttcatcagagacagtctggagctaattcttggtctctatcgccatgacaacctctcctctgctacctgggataccgacaagacggagcacttcctgttgtgcatccacagacagatagatggactcaatagctgtgtgagtagacagacagagagacacttcaactcctttcagctctttacttcatctgatacttaaagtgctttcagtgcttatacttcagtcaacagttcaactgacatcttttcacacatggacttacatcaactcctttcagtgcttacacttcaactgccagtttaacttctttgagtacgtacacatcagctgaaacacttaaaaacacaacttaaacttcaaatgtgctgaaattgtgctcagacatcaactggcactaaacttattttagtgcttacactttttatacttcagtcaacgcttcaactgacatatttgagtttttacacgtggactgactcctttcatgacttacacttcaacttctagctccttttaccacttacacctcaagcaacactttaactccttgtgtgacacaacagcccttctacttccgtcattacatttttacacttgaacagacaccaactactttcaggacttagcctgcaactcacacttcaaactcttttgacaaattacactttaagtgacatttcaactgacacttaaagtccttttagtacttacactttctagtgacatgtaacgccttgtatttccccgaacatatcaagtgacttcttatgcttcaaacttcaagctcctttcactacttcaactgtcatgtcaatgcctttcactgcttacacttttccttttacatgttcaacttcaggttttgcctccaaccacagacaaacaatctgacctgtgtttttgtcttcaggtgtcgactaacaagcaagcaaactacaaactgaaaaaatactacaagagactggagaaaagcactctgtaccgcactgtgagtacaagcactgttgtagtactgactgtagttctgtgtacagggtgaaagtattggtactgactgtagttctgtgtacagggtgaaagtagtagtactgactgtagttctgtgtacagggtggtagtgctgcgtcctgggagctgatcaggaaggaaactgaagtgcacctggaacggtcggccctgctggtggcccccatagctacaacacttcatcaccaacactgacacgtctcatcagtctgtctcactgtctccagcgtttattctgtctctctgtgagagtttctattgtcagtctatttatttatttatcatttatcgtttctatttatgaacaaagtgctcatcttgtatttatttatttattgaactctgacagttttgtatttacagactctgagtctgattttacttgaaaaattttgataacggaccacaataaagacatttcatacaaaaaaaaaaaaaaaaaaaaaaaaaaaaaaaaa

SEQ ID NO: 3 - DNA, 20 bp, Artificial Sequence
  Primer
agatcatgggcggtcagatg

SEQ ID NO: 4 - DNA, 20 bp, Artificial Sequence
  Primer
tgctacctgggataccgaca

SEQ ID NO: 5 - DNA, 20 bp, Artificial Sequence
  Primer
acactacggtcatgtgagca

SEQ ID NO: 6 - DNA, 21 bp, Artificial Sequence
  Primer
tccctcgctcttcaaatgctt

SEQ ID NO: 7 - DNA, 20 bp, Artificial Sequence
  Primer
cctgggataccgacaagacg

SEQ ID NO: 8 - DNA, 20 bp, Artificial Sequence
  Primer
aagcactctgtaccgcactg

SEQ ID NO: 9 - DNA, 25 bp, Artificial Sequence
  Primer
gactaacaagcaagcaaactacaaa

SEQ ID NO: 10 - DNA, 25 bp, Artificial Sequence
  Primer
cccccatagctacaacacttcatca

SEQ ID NO: 11 - DNA, 20 bp, Artificial Sequence
  Primer
agcatgttggagacagcgac

SEQ ID NO: 12 - DNA, 20 bp, Artificial Sequence
  Primer
gaagcttcctctgctcgtcc

(19) United States
(12) Patent Application Publication
(10) Pub. No.: US 2023/0332248 A1
(43) Pub. Date: Oct. 19, 2023

(54) SNP MOLECULAR MARKER RELATED TO SINIPERCA CHUATSI INFECTIOUS SPLEEN AND KIDNEY NECROSIS VIRUS RESISTANCE, DETECTION METHOD AND USE THEREOF

(71) Applicant: SOOCHOW UNIVERSITY, Suzhou (CN)

(72) Inventors: Hezhong HUANG, Suzhou (CN); Yuan XUE, Suzhou (CN); Zhongyi LU, Suzhou (CN); Miaomiao LIU, Suzhou (CN)

(21) Appl. No.: 17/800,754

(22) PCT Filed: May 21, 2021
(86) PCT No.: PCT/CN2021/095112

§ 371 Date: Aug. 18, 2022

(30) Foreign Application Priority Data
Apr. 30, 2021 (CN) 202110485520.6

(51) Int. Cl. C12Q 1/6888; C12Q 1/6869

(52) U.S. Cl. CPC C12Q 1/6888; C12Q 1/6869; C12Q 2600/156; C12Q 2600/124

(57) ABSTRACT

The invention relates to an SNP molecular marker located on a Siniperca chuatsi IFN-α3 gene and related to S. chuatsi infectious spleen and kidney necrosis virus (ISNKNV) resistance. On the basis of known cDNA, cloning is further carried out, an intron and a gDNA sequence of the S. chuatsi IFN-α3 gene are obtained, a primer is designed according to the gDNA sequence of IFN-α3, and through product amplification, and sequencing and alignment, SNP sites related to the disease-resistant or disease-susceptible characteristic of S. chuatsi germplasm are determined. Whereby, disease-resistant or disease-susceptible S. chuatsi germplasm can be rapidly screened out. The invention solves the problem of lack of the fish IFN-α3 gene, and also provides a theoretical basis and an operation method for breeding of disease-resistant germplasm by utilizing the heredity of the SNP sites and the correlation between the SNP sites and the disease-resistant characteristic.

### FIELD OF THE INVENTION

The invention relates to the fields of aquaculture and biotechnology, and specifically to an SNP molecular marker related to Siniperca chuatsi infectious spleen and kidney necrosis virus (ISNKNV) resistance, a detection method and use thereof.

### DESCRIPTION OF THE RELATED ART

Siniperca chuatsi belongs to the order Perciformes, the subfamily Sinipercinae, and the genus Siniperca. It is an important economic fish species in China, with its meat being delicious and rich in high protein and a variety of microorganisms and trace elements. However, due to the sharp decline of wild original germplasm resources, weak disease resistance, and germplasm degradation, infectious spleen and kidney necrosis virus (ISKNV) has become the most serious fulminant viral disease in Siniperca chuatsi farming, causing huge economic losses.

Conventional breeding technologies mainly rely on visual observation, which is difficult to implement, time-consuming and labor-intensive, and is likely to cause errors due to individual differences. Conventional ISKNV detection techniques mainly include histopathological detection, virus isolation, ELISA, conventional PCR, etc. Such detection methods rely on viral pathogens, require sensitive cell lines and complete laboratories, and are time-consuming and labor-intensive. Therefore, it is necessary to develop a novel germplasm detection method.

Signal nucleotide poly-morphisms (SNPs) are an effective molecular marker detection method. The insertion, deletion, conversion, or transversion of a single base can cause differences in transcription and translation processes, resulting in genetic differences in physiological functions such as disease resistance between individuals.

IFN-α3 (interferon-α3) belongs to fish type I interferon, which exerts antiviral activity through the JAK-STAT pathway and can establish an antiviral state in cells by inducing antiviral IsG. However, there are few related disease resistance studies on fish IFN-α3 gene. Therefore, it is of great significance to obtain the full-length gDNA sequence of Siniperca chuatsi IFN-α3 and explore the correlation between this gene and disease resistance.

### SUMMARY OF THE INVENTION

In order to solve the above-mentioned technical problems, the invention further carries out cloning on the basis of known cDNA and obtains an intron and a gDNA sequence of the Siniperca chuatsi IFN-α3 gene to solve the problem of lack of fish IFN-α3 gene, and further determines SNP sites related to the disease-resistant or disease-susceptible characteristic of Siniperca chuatsi germplasm, providing a basis and a new idea for the breeding of excellent Siniperca chuatsi varieties.

A first object of the invention is to provide a gDNA sequence of Siniperca chuatsi IFN-α3 gene, having a nucleotide sequence as shown in SEQ ID NO. 1.

A second object of the invention is to provide an SNP molecular marker located on a Siniperca chuatsi IFN-α3 gene and related to Siniperca chuatsi infectious spleen and kidney necrosis virus (ISNKNV) resistance, having a nucleotide sequence as shown in SEQ ID NO. 1 or SEQ ID NO. 2, where a 376bp site of the sequence has an A/G polymorphism.

Preferably, Siniperca chuatsi containing the SEQ ID NO. 1 sequence described above (where the 376th base is A) is susceptible to ISNKNV; and Siniperca chuatsi containing the SEQ ID NO. 2 sequence described above (where the 376th base is G) is resistant to ISNKNV.

Preferably, SNP sites of the SNP molecular marker are determined by the following steps:
(1) designing specific primers according to the gDNA sequence of the Siniperca chuatsi IFN-α3 gene, and obtain an amplification product through PCR amplification; and
(2) determining the SNP sites by extracting and detecting DNA of Siniperca chuatsi germplasm, amplifying the designed primers, sequencing the product, and carrying out comparison based on a DNA peak map and sequence.

Preferably, in the step (2), multiple sequence alignment is carried out on the sequencing result using DNAMAN software, suspected SNPs sites are found, and the DNA peak map is checked using Chromas software to determine whether there are overlapping peaks, to determine the SNP sites.

The invention provides use of the SNP molecular marker described above in the preparation of a reagent for identification or screening of Siniperca chuatsi infectious spleen and kidney necrosis virus (ISNKNV) resistance.

The invention provides use of the SNP molecular marker described above in the preparation of a reagent for breeding of Siniperca chuatsi germplasm.

The invention also provides use of the SNP molecular marker described above in improving Siniperca chuatsi infectious spleen and kidney necrosis virus (ISNKNV) resistance.

A third object of the invention is to provide a primer pair for detecting the SNP molecular marker described above, having nucleotide sequences as shown in SEQ ID NO. 11 and SEQ ID NO. 12, wherein specifically,

IFN-α3-1F:
5′-AGCATGTTGGAGACAGCGAC-3′;
and
IFN-α3-1R:
5′-GAAGCTTCCTCTGCTCGTCC-3′.

A fourth object of the invention is to provide a kit for detecting the SNP molecular marker described above, including the primer pair described above.

The invention provides an use of the primer pair or kit described above in the preparation of a reagent for identification or screening of Siniperca chuatsi infectious spleen and kidney necrosis virus (ISNKNV) resistance.

The invention also provides use of the primer pair or kit described above in the preparation of a reagent for breeding of Siniperca chuatsi germplasm or in improving Siniperca chuatsi infectious spleen and kidney necrosis virus (ISNKNV) resistance.

A fifth object of the invention is to provide a germplasm breeding method for Siniperca chuatsi with resistance to infectious spleen and kidney necrosis virus (ISNKNV), including the following steps:
(1) extracting a genomic DNA of Siniperca chuatsi to be tested;
(2) performing PCR amplification on the genomic DNA of the Siniperca chuatsi using the primer pair or the kit described above to obtain a PCR amplification product; and
(3) sequencing the PCR amplification product, comparing a result of the sequencing with the SNP molecular marker described above, determining whether a base at a position corresponding to the result of the sequencing is A or G, and eliminating the Siniperca chuatsi with the base A at the corresponding position and maintaining the Siniperca chuatsi with the base G at the corresponding position, to obtain the Siniperca chuatsi with resistance to ISNKNV.

By virtue of the above solutions, the invention has the following advantages.
(1) The invention clones the gDNA full-length sequence of the Siniperca chuatsi IFN-α3 gene for the first time, which lays a foundation for the breeding of fish germplasm.
(2) The invention develops a method for detecting Siniperca chuatsi germplasm with resistance to ISKNV, so that the SNP sites of the Siniperca chuatsi IFN-α3 gene correlated to ISKNV resistance can be detected, which provides a new idea for Siniperca chuatsi farming and improves economic value.

The above description is only a summary of the technical solutions of the invention. To make the technical means of the invention clearer and implementable in accordance with the disclosure of the specification, the preferred embodiments of the invention will be described in detail with reference to the accompanying drawings.

### BRIEF DESCRIPTION OF THE DRAWINGS

To make the content of the invention more comprehensible, the invention will be described in further detail below according to specific embodiments of the invention and in conjunction with the accompanying drawings, wherein:

FIG. 1 shows the results of gel electrophoresis after PCR amplification of the Siniperca chuatsi IFN-α3 gene with the addition of a primer for the intron;

FIG. 2 shows the results of gel electrophoresis after SNP site detection and PCR amplification of the Siniperca chuatsi IFN-α3 gene;

FIG. 3 is a screenshot of the results of comparison of IFN-α3 DNA sequences of 16 Siniperca chuatsi samples, where base A is mutated to base G in five samples;

FIG. 4 is a DNA peak map generated by sequencing of IFN-α3 DNA of a Siniperca chuatsi sample using a gene sequencing instrument, where in the IFN-α3 DNA of the Siniperca chuatsi sample in the figure,the site 297 was mutated to G homozygote;

FIG. 5 is a DNA peak map generated by sequencing of IFN-α3 DNA of a Siniperca chuatsi sample using a gene sequencing instrument, where in the IFN-α3 DNA of the Siniperca chuatsi sample in the figure, the site 297 was A homozygote; and

FIG. 6 is a DNA peak map generated by sequencing of IFN-α3 DNA of a Siniperca chuatsi sample using a gene sequencing instrument, where in the IFN-α3 DNA of the Siniperca chuatsi sample in the figure, the site 297 was A heterozygote.

### DETAILED DESCRIPTION OF THE PREFERRED EMBODIMENTS

The invention will be further described below in conjunction with the accompanying drawings and specific embodiments, so that those skilled in the art can better understand and implement the invention, but the embodiments described are not intended to limit the invention.

### Example 1 Amplification of the Full-Length Sequence of the Siniperca chuatsi IFN-α3 Gene

### 1. Amplification of Intron of Siniperca chuatsi IFN-α3 Gene

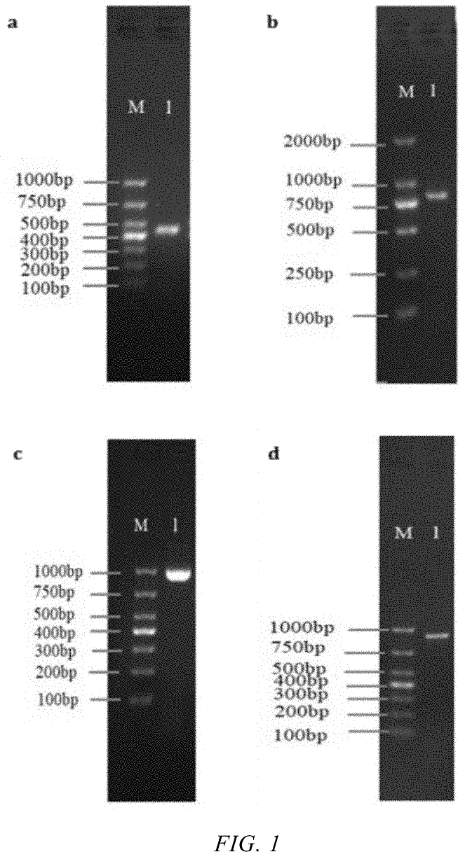

Specific primers were designed with reference to the complete fragment of the known Siniperca chuatsi IFN-α3 cDNA to amplify the intron of the IFN-α3 gene (see Table 1). The Siniperca chuatsi genome DNA was used as a template for fragmented PCR amplification of the gene. The amplification procedure is shown in Table 2. The PCR products were sequenced. The obtained sequences were spliced by manual alignment and DNAMAN software to obtain the full length of the Siniperca chuatsi IFN-α3 gDNA gene, as shown in SEQ ID NO. 1. The results of gel electrophoresis after PCR amplification of the Siniperca chuatsi IFN-α3 gene with the addition of the primer for the intron are shown in FIG. 1, where a is an electrophoretogram of an amplification product of IFN-α3-4F,4R, b is an electrophoretogram of an amplification product of IFN-a3-3F,3R, c is an electrophoretogram of an amplification product of IFN-a3-1F,1R, and d is an electrophoretogram of an amplification product of IFN-a3-2F,2R.

TABLE 1
Sequences of primers used to amplify
the intron of the IFN-α3 gene

| Primer name | Primer sequence (5′-3′) |
|---|---|
| IFN-α3-1F | AGATCATGGGCGGTCAGATG |
| IFN-α3-1R | TGCTACCTGGGATACCGACA |
| IFN-α3-2F | ACACTACGGTCATGTGAGCA |
| IFN-α3-2R | TCCCTCGCTCTTCAAATGCTT |
| IFN-α3-3F | CCTGGGATACCGACAAGACG |
| IFN-α3-3R | AAGCACTCTGTACCGCACTG |
| IFN-α3-4F | GACTAACAAGCAAGCAAACTACAAA |
| IFN-α3-4R | CCCCCATAGCTACAACACTTCATCA |

TABLE 2
Amplification procedure

| Procedure name | Temperature | Time | |
|---|---|---|---|
| Pre-denaturization | 94° C. | 5 | min |
| Denaturization | 94° C. | 30 | s |
| Anneal | 52° C. | 30 | s |
| Extension | 72° C. | 1 | min |

×35 cycle

| | | | |
|---|---|---|---|
| Final extension | 72° C. | 10 | min |

### 2. Detection of Disease-Resistant SNP Marker

(1) Primers were designed by using the sequence of Siniperca chuatsi IFN-α3 gDNA as a template (see Table 3), and PCR amplification was carried out. The reaction system is shown in Table 4. The reaction procedure is shown in Table 5.

TABLE 3
Primer sequences for amplification of
Siniperca chuatsi IFN-α3 gene to
obtain disease-resistant SNP sites

| Primer name | Primer sequence (5′-3′) |
|---|---|
| IFN-α3-SNP-1F | AGCATGTTGGAGACAGCGAC |
| IFN-α3-SNP-1R | GAAGCTTCCTCTGCTCGTCC |

TABLE 4
PCR reaction system for amplification of Siniperca chuatsi
IFN-α3 gene to obtain disease-resistant SNP sites

| Component | Volume (μL) |
|---|---|
| Template (DNA) | 2 |
| Primer R (10 μL) | 1 |
| Primer F (10 μL) | 1 |
| 2 × Easy Taq PCR Super Mix | 25 |
| ddH₂O | 21 |
| In total | 50 |

TABLE 5
PCR reaction procedure for amplification of Siniperca chuatsi
IFN-α3 gene to obtain disease-resistant SNP sites

| Procedure name | Temperature | Time | |
|---|---|---|---|
| Pre-denaturization | 94° C. | 5 | min |
| Denaturization | 94° C. | 30 | s |
| Anneal | 53° C. | 30 | s |
| Extension | 72° C. | 1 | min |

×35 cycle

| | | | |
|---|---|---|---|
| Final extension | 72° C. | 10 | min |

(2) The PCR products were detected by 1.5% agarose gel electrophoresis. The PCR product with obvious lanes was selected and sequenced.

(3) Multiple sequence alignment was carried out using DNAMAN software, SNPs sites correlated to the virus resistance were found, and the DNA peak map was checked using Chromas software to determine the SNP sites.

### Example 2 Detection Results Based on Disease-Resistant SNP Markers of Siniperca chuatsi IFN-α3 Gene

Siniperca chuatsi in the same group were cultured according to the same feeding conditions. After about two months of culture, 100 fishes were randomly selected from the cultured group for a challenge test. Each fish in each group was given 200 μL of infectious spleen and kidney necrosis virus (ISKNV, also known as iridovirus) (10^{3.68} TCID50/mL) by intraperitoneal injection, and observed for 10 consecutive days. The observation showed that the symptoms of some fishes were the same as those of fished infected with ISKNV in the natural environment within the 10 days, the diseased fishes swam slowly on the water surface or even directly floated on the water surface, with the body surface being not damaged, the body color being white, and the fish gills being ischemic white. The anatomy showed that: there was a lot of ascites in the abdominal cavity; the liver, stomach wall, and intestinal wall were engorged with blood, and yellow fluid was found in the intestine. These fishes were determined to be disease-susceptible Siniperca chuatsi. The fishes exhibiting no abnormality on the 10th day were determined to be disease-resistant Siniperca chuatsi. Siniperca chuatsi whose conditions cannot be judged were not used as samples. PCR-based detection showed that the head kidney of the diseased Siniperca chuatsi was positive for ISKNV, indicating that the cause of death was due to infection with ISKNV.

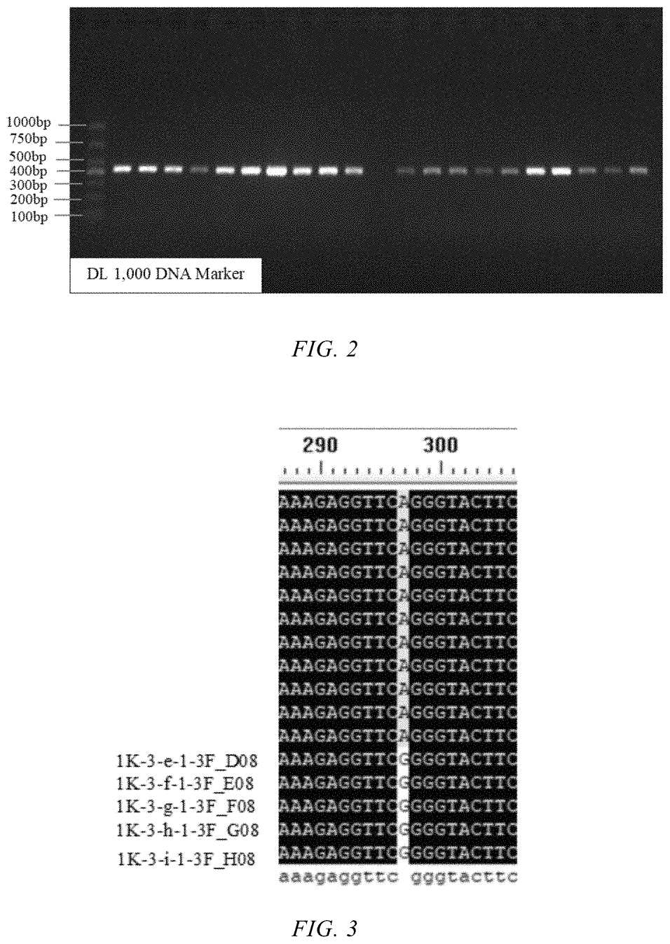

12 fishes of disease-susceptible Siniperca chuatsi and 8 fishes of disease-resistant Siniperca chuatsi were selected respectively (with the disease-resistant Siniperca chuatsi being marked as A08, B08, C08, D08, E08, F08, G08, and H08), from which a small amount of tail fin was cut off and put into absolute ethanol for cryopreservation at 4° C. PCR amplification was carried out according to the disease-resistant SNP marker detection method in the above example. The result of electrophoresis is shown in FIG. 2. After sequencing of 20 samples, clear and complete nucleic acid sequences of 11 disease-susceptible Siniperca chuatsi samples and 5 disease-resistant Siniperca chuatsi samples were obtained. Multiple sequence alignment was carried out using DNAMAN software, and SNP sites correlated to disease resistance were found at the 297 bp in this nucleic acid fragment, as shown in FIG. 3.

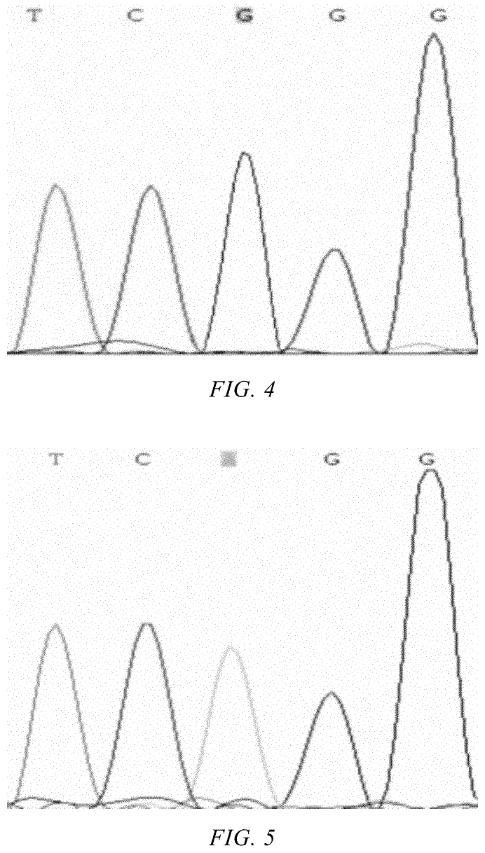

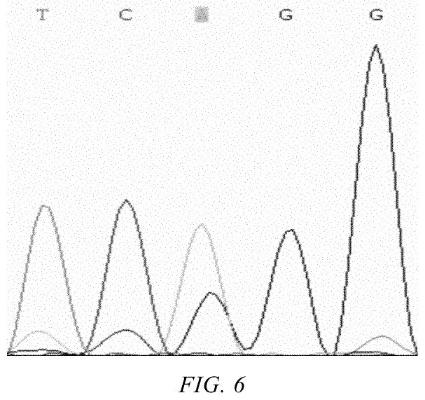

Peak maps where the suspected mutated base in the above sequence is at 297 bp in the nucleic acid fragment were checked using Chromas software, as shown in FIG. 4, FIG. 5, and FIG. 6. The peaks obtained at the bases G and A corresponding to each other are all single peaks obviously without any stray peaks. Therefore, it can be concluded that the base is mutated at 297 bp of the nucleic acid fragment, and is converted from A to G, that is, A297G. As can be seen from comparison with the original DNA sequence, the mutation occurred at 376 bp of the original DNA sequence, which is located in the intron, where the base at the 376 bp is A for disease-susceptible Siniperca chuatsi and is G for disease-resistant Siniperca chuatsi, that is, the SNP site is A376G. The 5 samples with base mutation (D08, E08, F08, G08, H08) were all disease-resistant Siniperca chuatsi. The base was not mutated in disease-susceptible Siniperca chuatsi. Therefore, the SNP of the IFN-α3 gene of the disease-resistant Siniperca chuatsi is a mutation of the base from A to G at 376 bp. By this method, the disease-resistant Siniperca chuatsi and the disease-susceptible Siniperca chuatsi can be identified.

Apparently, the above-described embodiments are merely examples provided for clarity of description, and are not intended to limit the implementations of the invention. Other variations or changes can be made by those skilled in the art based on the above description. The embodiments are not exhaustive herein. Obvious variations or changes derived therefrom also fall within the protection scope of the invention.

1. An SNP molecular marker related to Siniperca chuatsi infectious spleen and kidney necrosis virus (ISNKNV) resistance, wherein a nucleotide sequence of the SNP molecular marker is as shown in SEQ ID NO. 1 or SEQ ID NO. 2.

2. The SNP molecular marker according to claim 1, wherein Siniperca chuatsi containing the SEQ ID NO. 1 sequence is susceptible to ISNKNV; and S. chuatsi containing the SEQ ID NO. 2 sequence is resistant to ISNKNV.

3. Use of the SNP molecular marker of claim 1 in the preparation of a reagent for identification or screening of Siniperca chuatsi infectious spleen and kidney necrosis virus (ISNKNV) resistance.

4. Use of the SNP molecular marker of claim 1 in the preparation of a reagent for breeding of Siniperca chuatsi germplasm.

5. Use of the SNP molecular marker of claim 1 in improving Siniperca chuatsi infectious spleen and kidney necrosis virus (ISNKNV) resistance.

6. A primer pair for detecting the SNP molecular marker of claim 1, wherein nucleotide sequences of the primer pair are as shown in SEQ ID NO. 11 and SEQ ID NO. 12.

7. A kit for detecting the SNP molecular marker of claim 1, wherein the kit comprises the primer pair of claim 6.

8. Use of the primer pair of claim 6 in the preparation of a reagent for identification or screening of Siniperca chuatsi infectious spleen and kidney necrosis virus (ISNKNV) resistance.

9. Use of the primer pair of claim 6 in the preparation of a reagent for breeding of Siniperca chuatsi germplasm or in improving S. chuatsi infectious spleen and kidney necrosis virus (ISNKNV) resistance.

10. A germplasm breeding method for Siniperca chuatsi with resistance to infectious spleen and kidney necrosis virus (ISNKNV), comprising steps of:
(1) extracting a genomic DNA of S. chuatsi to be tested;
(2) performing PCR amplification on the genomic DNA of the Siniperca chuatsi using the primer pair of claim 6 to obtain a PCR amplification product;
(3) sequencing the PCR amplification product, comparing a result of the sequencing with an SNP molecular marker, wherein a nucleotide sequence of the SNP molecular marker is as shown in SEQ ID NO. 1 or SEQ ID NO. 2, determining whether a base at a position corresponding to the result of the sequencing is A or G, and eliminating the Siniperca chuatsi with the base A at the corresponding position and maintaining the S. chuatsi with the base G at the corresponding position, to obtain the S. chuatsi with resistance to ISNKNV.
